(19) United States
(12) Patent Application Publication
(10) Pub. No.: US 2004/0043073 A1
(43) Pub. Date: Mar. 4, 2004

(54) Pharmaceutical compositions for drugs having pH-dependent solubility

(76) Inventors: Chih-Ming Chen, Taipei (TW); Boyong Li, Morgantown, WV (US); Avinash Nangia, Weston, FL (US)

Correspondence Address: DAVIDSON, DAVIDSON & KAPPEL, LLC, 14th Floor, 485 Seventh Avenue, New York, NY, 10018, US

(21) Appl. No.: 10/462,581

(22) Filed: Jun. 16, 2003

Related U.S. Application Data
(60) Provisional application No. 60/388,704, filed on Jun. 14, 2002.

(51) Int. Cl. A61K 31/7048; A61K 9/14

(52) U.S. Cl. 424/486; 514/29

(57) ABSTRACT

The present invention is directed to oral pharmaceutical formulations containing drugs having pH dependent solubility and which are suitable for administration to a patient in need of treatment related thereto, and methods of preparing the same.

This application claims the benefit of U.S. Provisional Application Serial No. 60/388,704, the disclosure of which is hereby incorporated by reference.

### FIELD OF THE INVENTION

The invention is directed to oral pharmaceutical formulations containing drugs having pH-dependent solubility and methods of preparation and treatment thereof. In certain preferred embodiments, the present invention relates to immediate or controlled release oral dosage forms comprising clarithromycin, pharmaceutically acceptable salts thereof, or active metabolites thereof.

### BACKGROUND OF THE INVENTION

It is known in the pharmaceutical art to prepare compositions which provide for immediate or controlled release of pharmacologically active substances contained in the compositions after oral administration to humans and animals. Such compositions can be used, e.g., to provide substantially immediate bioavailability of the active agent or to delay absorption of the active agent until it has reached certain portions of the alimentary tract.

It is typically the goal of a controlled-release preparation to provide a longer period of pharmacologic response after the administration of the dosage form than that which is ordinarily experienced after the administration of an immediate release dosage form. Preferably a controlled release dosage form should deliver the active agent at a constant rate throughout the gastrointestinal tract. As different drugs have different physical and chemical properties, this expectation may not be realized with many of the delivery systems currently available. For example, all controlled release technologies may not be suitable for drugs having a pH dependent solubility which varies in relation to the pH within the body. The pH fluctuations within the body may result in a decreased release rate of the drug. Additionally, the pH fluctuations may result in a decreased bioavailablity of the drug.

One factor which must be considered in formulating an active agent in a controlled release or immediate release preparation is the stability of the agent in an environment of use, e.g., the gastric system.

In view of the aforementioned, there exists a need in the art to provide both immediate release and controlled release formulations for drugs having pH dependent solubility.

### SUMMARY OF THE INVENTION

It is an object of certain embodiments of the present invention to provide a pharmaceutical dosage form providing a controlled release of a drug having a pH dependent solubility.

It is an object of certain embodiments of the present invention to provide a pharmaceutical dosage form comprising a therapeutically effective amount of a drug having a pH dependent solubility and at least one inorganic pH modifying agent.

It is an object of certain embodiments of the present invention to provide a pharmaceutical dosage form comprising a therapeutically effective amount of drug having a pH dependent solubility and at least one inorganic pH modifying agent which is not substantially effected by physiological pH changes.

It is an object of certain embodiments of the present invention to provide a controlled release oral dosage form comprising a therapeutically effective amount of a drug having a pH dependent solubility, a pH modifier, and a wax material, and methods of preparation of the same.

It is an object of certain embodiments of the present invention to provide for a controlled release matrix comprising a therapeutically effective amount of drug having a pH dependent solubility, a wax material, and a pH modifier which results in a dosage form which is responsive to physiological pH changes.

It is an object of certain embodiments of the present invention to provide a pharmaceutical dosage form comprising a therapeutically effective amount of a drug having a pH dependent solubility wherein the drug is stabilized upon exposure to an environmental fluid.

In certain embodiments of the present invention, the drug having a pH dependent solubility is a macrolide antibiotic selected from the group consisting of azithromycin, clarithromycin, dirithromycin, erythromycin, troleandomycin, pharmaceutically acceptable salts thereof, active metabolites thereof, derivatives thereof and mixtures thereof. In certain embodiments, the active metabolite is 14-OH clarithromycin.

In certain embodiments of the present invention, the dosage form comprises a drug having a pH dependent solubility and an effective amount of at least one inorganic pH modifying agent selected from the group consisting of sodium phosphate monobasic, hydrated forms thereof, potassium phosphate monobasic, hydrated forms thereof, and mixtures thereof.

In certain embodiments of the present invention, the dosage form is an oral dosage form such as a tablet, a capsule or any other suitable dosage form known in the art.

In certain embodiments of the present invention, the drug and the inorganic pH modifying agent are subjected to a wet or dry granulation.

In certain embodiments of the present invention, the dosage form provides a bioavailability which is from 80% to 125% of the bioavailability of a reference standard, e.g., Biaxin® XL, Biaxin® Filmtab® or Biaxin® Granules.

In certain embodiments of the present invention, the oral dosage form provides for the immediate or controlled release of the drug from the dosage form.

In certain embodiments of the present invention, the dosage form comprises a sufficient amount of a polymeric material to provide a controlled release of the drug for at least 12 or at least 24 hours in an environment of use.

In certain embodiments of the present invention, the controlled release polymeric material is a cellulosic material or an acrylic polymer.

In certain embodiments of the present invention providing a controlled release, the controlled release oral dosage form comprising a matrix comprising a drug having a pH dependent solubility; at least one wax material in an effective amount to provide a controlled release of said drug for at least 12 hours in an environment of use; and at least one pH modifying agent.

In certain embodiments, the present invention is directed to a controlled release oral dosage form comprising clarithromycin or a pharmaceutically acceptable salt thereof and at least one controlled release excipient to provide a controlled release of said clarithromycin for at least 12 hours after administration to a human patient, wherein said dosage form provides a mean AUC under fasted conditions which does not differ from the mean AUC under fed conditions by more than plus or minus 10%.

In certain embodiments, the invention is directed to a method of preparing a controlled release oral dosage by wet granulation; said controlled release dosage form comprising a drug having a pH dependent solubility; a least one wax material; and at least one pH modifying agent.

In certain embodiments, the invention is directed to a pharmaceutical dosage form comprising a drug having a pH dependent solubility; and at least one inorganic compound in an effective amount to stabilize said drug.

In certain embodiments, the invention is directed to a controlled release pharmaceutical dosage form comprising a macrolide antibiotic and a sufficient amount of a wax material to provide a controlled release of said macrolide antibiotic.

In certain embodiments, the invention is directed to a controlled release pharmaceutical dosage form comprising a therapeutically active agent and a sufficient amount of glycerol monostearate to provide a controlled release of the agent. In such embodiments, the agent is without limitation and can be any agent which provides a therapeutic effect, but is preferably an antibiotic, more preferably a macrolide antibiotic and most preferably clarithromycin or a pharmaceutically acceptable salt thereof. In such embodiments, the glycerol monostearate can be in the dosage form in an amount of about 10% or greater, about 15% or greater, about 25% or greater or about 50% or greater, e.g., from about 10% to about 60%, from about 20% to about 40%, from about 10% to about 30% or from about 15% to about 25%.

In certain embodiments, the invention is directed to a controlled release pharmaceutical dosage form comprising a macrolide antibiotic; a sufficient amount of a polymeric material to provide a controlled release of said macrolide antibiotic; and an effective amount of at least one inorganic compound to stabilize said macrolide antibiotic.

In certain embodiments, the invention is directed to a controlled release pharmaceutical dosage form comprising a macrolide antibiotic; a sufficient amount of a polymeric material to provide a controlled release of said macrolide antibiotic; and an effective amount of at least one organic compound to stabilize said macrolide antibiotic, said organic compound selected from the group consisting of citric acid, succinic acid, fumaric acid, malic acid, maleic acid, glutaric acid, lactic acid.

In certain embodiments the drug is a macrolide antibiotic, e.g., an erythromycin derivative such as clarithromycin or a pharmaceutically acceptable salt thereof, and provides a method for treating a microbial infection in a mammal which comprises administering to a mammal that is in need of such treatment, an antimicrobially effective amount of said macrolide antibiotic in a controlled release oral dosage form described herein.

In certain embodiments, the dosage form of the present invention can provide a substantially pH-independent in-vitro dissolution rate.

In certain embodiments, the pH modifier of the present invention can provide a pH in a micro environment of greater than 3 to less than 7.

In certain embodiments, the dosage form can provide therapeutic levels of drug for at least 12 or at least 24 hours.

In certain embodiments wherein the active agent is clarithromycin or a pharmaceutically acceptable salt thereof, the dosage form can provide a mean AUC under fasted conditions which does not differ from the mean AUC under fed conditions by more than plus or minus 10%.

In alternate embodiments, the present invention is directed to a controlled release pharmaceutical dosage form comprising a macrolide antibiotic and a sufficient amount of a wax material to provide a controlled release of the macrolide antibiotic.

In alternate embodiments, the present invention is directed to a controlled release pharmaceutical dosage form comprising an active agent and a sufficient amount of glycerol monostearate to provide a controlled release of the agent.

In alternate embodiments, the present invention is directed to a controlled release pharmaceutical dosage form comprising a macrolide antibiotic; a sufficient amount of a polymeric material to provide a controlled release of the macrolide antibiotic; and an effective amount of at least one compound selected from the group consisting of an organic compound and an inorganic compound to stabilize the drug.

In certain embodiments, the invention is directed to a method of preparing a pharmaceutical dosage form comprising combining at least one drug having a pH dependent solubility with at least one inorganic stabilizing agent and at least one wax material in an effective amount to provide a controlled release of the drug for at least 12 hours in an environment of use. In certain embodiments, the drug, inorganic stabilizing agent and wax material are wet or dry granulated and incorporated into a dosage form.

In certain embodiments, the invention is directed to a method of preparing a pharmaceutical dosage form comprising combining a macrolide antibiotic and a sufficient amount of a wax material to provide a controlled release of the macrolide antibiotic into a dosage form. In certain embodiments, the macrolide antibiotic and wax material are wet or dry granulated and incorporated into a dosage form.

In certain embodiments, the invention is directed to a method of preparing a pharmaceutical dosage form comprising combining an active agent and a sufficient amount of glycerol monostearate to provide a controlled release of the active agent into the dosage form. In certain embodiments, the active agent and glycerol monostearate are wet or dry granulated and incorporated into a dosage form.

In certain embodiments, the invention is directed to a method of preparing a pharmaceutical dosage form comprising combining a macrolide antibiotic, a sufficient amount of a polymeric material to provide a controlled release of the antibiotic and an effective amount of at least one compound to stabilize the drug into a dosage form. In certain embodiments, the macrolide antibiotic, polymeric material and stabilizer are wet or dry granulated and incorporated into a dosage form.

The term “dosage form” as it is used herein means a single dose contained in at least one unit dosage form of the present invention (e.g., the daily dose of clarithromycin can be contained in 2 unit dosage forms of the present invention for single once-a-day administration).

The term “erythromycin derivative” as it is used herein, means erythromycin having no substituent groups, or having conventional substituent groups, in organic synthesis, in place of a hydrogen atom of the hydroxy groups and/or a methyl group of the 3′-dimethylamino group, which is prepared according to the conventional manner, and pharmaceutically acceptable salts thereof.

The term “Cₘₐₓ” is the highest plasma concentration of the drug attained within the dosing interval.

The term “Tₘₐₓ” as it is used herein is the time period which elapses after administration of the dosage form until the plasma concentration of the drug attains the highest plasma concentration within the dosing interval.

The term “AUC₀₋₂₄” as used herein, means area under the plasma concentration-time curve, as calculated by the trapezoidal rule over the complete 24-hour interval.

The term “mean”, when preceding a pharmacokinetic value (e.g. mean Tₘₐₓ) represents the arithmetic mean value of the pharmacokinetic value taken from a population of patients unless otherwise specified.

Reference herein to the administration of the dosage form to a mammal (including humans) in a “fed” state means that the mammal has eaten food (e.g., a high fat meal as defined by the U.S. Food and Drug Administration) within one hour prior to dosing and/or up to two hours after dosing.

The term “pH modifying agent” is meant for purposes of the present invention to mean any substance which modifies the ionization of the drug, whereby the release of the drug from the dosage form and into solution is facilitated. In preferred embodiments, the pH modifying agent stabilizes the drug in an environment of use.

The term “stabilizer” is meant for purposes of the present invention to mean any substance included in a formulation which reduces the degradation of the drug upon exposure to an environmental fluid, as compared to the formulation without the inclusion of the stabilizer. In preferred embodiments, the stabilizer is also a pH modifying agent.

### BRIEF DESCRIPTION OF THE DRAWINGS

The following drawings are illustrative of embodiments of the invention and are not meant to limit the scope of the invention.

FIG. 1 is a graph of the effect of NaH₂PO₄ on degradation of clarithromycin granules in 0.01N HCl (pH 2.01).

FIG. 2 is a graph of the effect of NaH₂PO₄ on dissolution of clarithromycin granules in purified water (pH 6.80).

FIG. 3 is a graph of the effect of NaH₂PO₄ on dissolution of clarithromycin granules in 0.1N HCl (pH 2.01).

FIG. 4 is a graph of the effect of NaH₂PO₄ on dissolution of clarithromycin ER tablets in SIF(pH6.8)/0.5% Tween 80.

FIG. 5 is a graph of in vitro dissolution data which shows the dissolution profiles of the formulations Example 6 and reference standard Biaxin® XL in 0.1M NaAc at pH 5 in a USP Type I dissolution apparatus at 100 rpm.

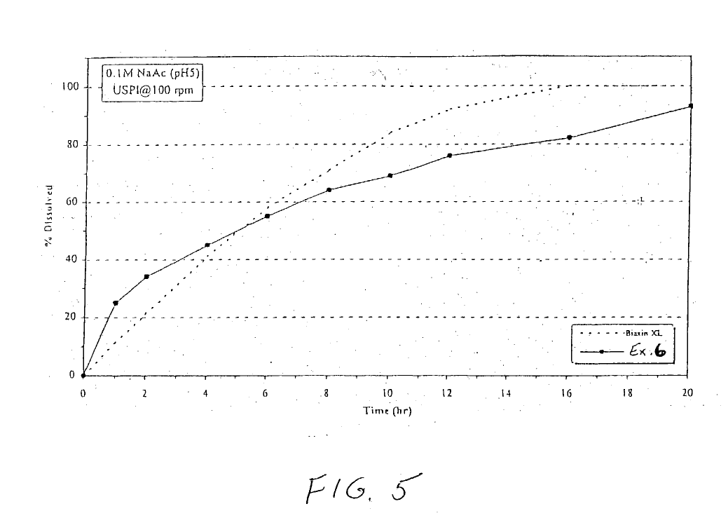

FIG. 6 is a graph of in vitro dissolution data which shows the dissolution profiles of the formulations Example 6 and reference standard Biaxin® XL in Simulated Intestinal Fluid at pH 6.8 with 0.50% Tween 80 in a USP Type I dissolution apparatus at 100 rpm.

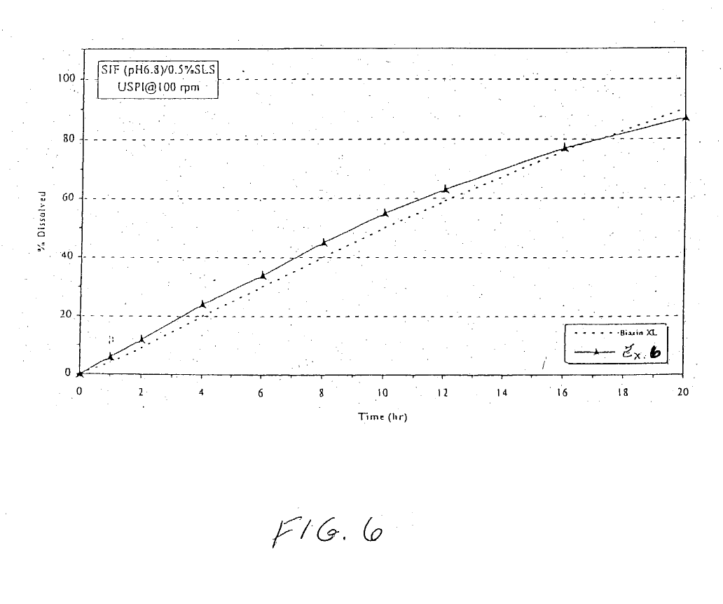

FIG. 7 is a graph of in vivo data which shows plasma concentrations curves of the formulations Example 6 and reference standard Biaxin® XL administered in the fasted state.

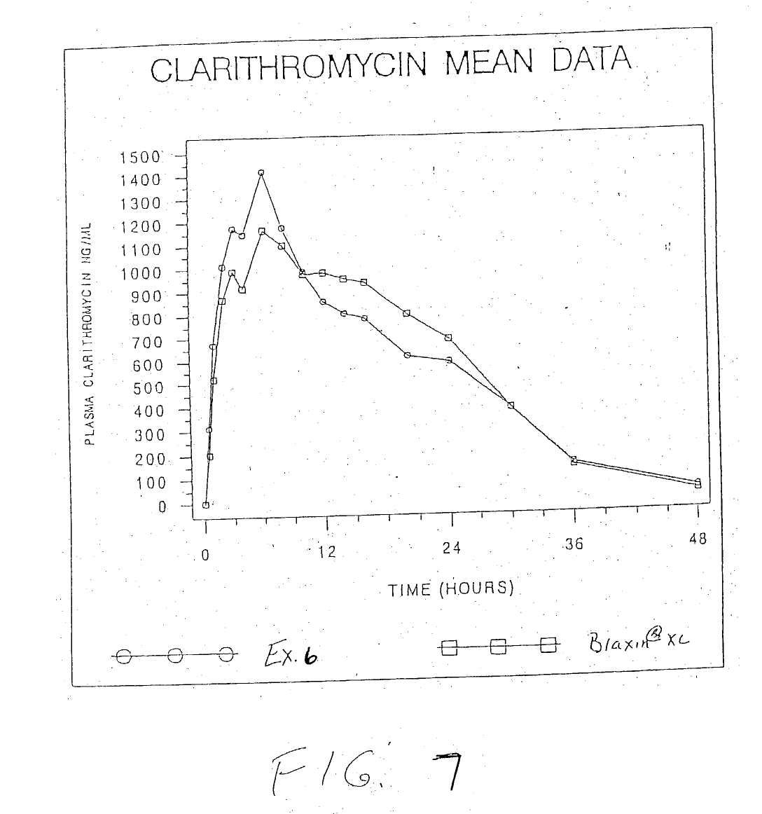

FIG. 8 is a graph of in vivo data which shows plasma concentrations curves of the formulations Example 6 and reference standard Biaxin® XL administered in the fed state.

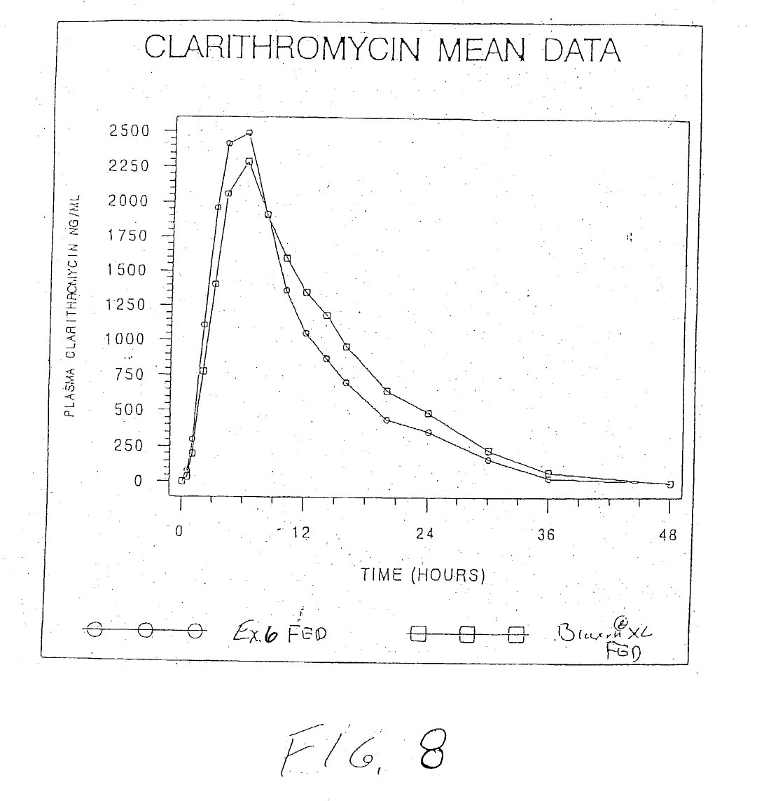

FIG. 9 is a graph of in vitro dissolution data which shows the dissolution profiles of the formulations of Example 6, Example 7, and Example 16.

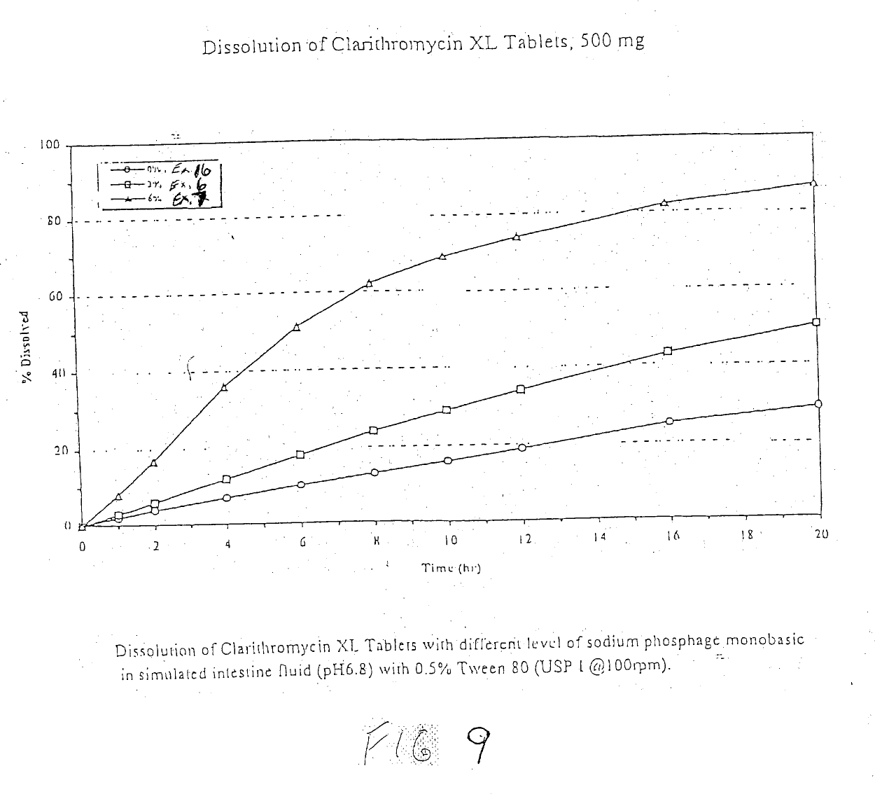

### DETAILED DESCRIPTION

In certain preferred embodiments of the present invention, the drug having a pH dependent solubility is a macrolide antibiotic. Macrolide antibiotics are typically used for the treatment of a wide range of bacterial infections. The class of macrolide antibiotics are compounds which typically include a 14-membered macrolactone ring and two O-linked sugar molecules. Examples of such macrolides are erythromycin, clarithromycin, dirithromycin, josamycin, midecamycin, kitasamycin, tylosin, roxithromycin, rokitamycin, oleandomycin, miocamycin, flurithromycin, rosaramicin, azithromycin, derivatives thereof, and pharmaceutically acceptable salts thereof.

The most preferred macrolide antibiotic for the present invention is clarithromycin, having a solubility of about one part in 1,000 parts of water. It is known that clarithromycin is very soluble in the stomach (pH 1.2) and fairly soluble in the upper region of the small intestine (pH 5.0) where absorption is most likely to occur. Because the drug's solubility decreases in the lower small intestine (pH 6 to 8), this leads to less drug being available for absorption. The present invention can provide a way of overcoming this problem by using a wax material with a pH modifying agent.

In certain preferred embodiments of the present invention, the controlled-release oral dosage form of the present invention includes from about 50 to about 1000 mg clarithromycin, and more preferably from about 250 mg to about 500 mg clarithromycin.

The macrolide antibiotic, 6-O-methylerythromycin A (clarithromycin), is particularly useful in treating common pediatric infections of the middle ear and upper respiratory tract. Other uses of clarithromycin are listed in the 54^{th} Edition of the Physicians' Desk Reference, copyright 2000, pp. 409-417, the disclosure of which is herein incorporated by reference.

Certain preferred embodiments of the present invention provide for a once daily dosage form of clarithromycin.

The present invention provides for a controlled release (e.g., once-a-day dosing) of at least one drug having a pH dependent solubility. More particularly, the present invention provides a controlled release pharmaceutical composition comprising a matrix that comprises a therapeutically effective amount of a drug having a pH dependent solubility; a wax material in an effective amount to provide a controlled release of the drug; and a pH modifying agent; such that the dosage form delivers the drug having a dependent solubility over an extended period of time.

In certain embodiments, the present invention relates to a dosage form comprising a controlled release matrix comprising from about 1% by weight to about 90% by weight of an active agent, from about 5% by weight to about 95% by weight of a wax material, and from about 0.1% by weight to about 25% by weight of a pH modifying agent. The release of the active agent from the formulation is not substantially affected by an increase in pH resulting in an improved controlled delivery of drugs whose solubility declines as the pH is increased.

In certain embodiments, the matrix comprises from about 20% by weight to about 75% by weight of an active agent, from about 5% by weight to about 95% by weight of a wax material, and from about 0.5% by weight to about 25% by weight of a pH modifying agent.

In certain embodiments, the matrix comprises from about 30% by weight to about 50% by weight of an active agent, from about 10% by weight to about 35% by weight of a wax material, and from about 1% by weight to about 8% by weight of a pH modifying agent.

In certain embodiments, the dosage form of the present invention can provide a mean time to maximum plasma concentration (Tₘₐₓ) of the drug at from about 1 hour to about 12 hours after administration, more preferably at from about 2 to about 10 hours after administration, and most preferably at from about 2 to about 8 hours after administration or at from about 4 to about 6 hours after administration.

In certain embodiments when the active agent is clarithromycin or a pharmaceutically acceptable salt thereof, the dosage form provides a bioavailability which is from 80% to 125% of the bioavailability of a reference standard (Biaxin® XL).

In certain embodiments, the dosage form can provide a mean AUC of from about 15 μg·h/ml to about 35 μg·h/ml based on administration of 500 mg clarithromycin; a mean AUC of from about 20 μg·h/ml to about 30 μg·h/ml based on administration of 500 mg clarithromycin; or a mean AUC of from about 22 μg·h/ml to about 28 μg·h/ml based on administration of 500 mg clarithromycin.

In certain embodiments, the dosage form of the present invention provides a mean Cₘₐₓ from about 1 μg/ml to about 2 μg/ml based on administration of 500 mg clarithromycin under fasting conditions and/or a mean Cₘₐₓ from about 2 μg/ml to about 3 μg/ml based on administration of 500 mg clarithromycin under fed conditions. Preferably, the controlled release oral dosage form provides a mean AUC for clarithromycin under fasted conditions which does not differ from the mean AUC for clarithromycin under fed conditions by more than plus or minus 10%.

In certain embodiments, the present invention provides for a pharmaceutical dosage form comprising a drug having a pH dependent solubility; and an effective amount of at least one inorganic pH modifying agent to stabilize the drug. The dosage form can provide an immediate release (e.g., multiple daily dosing) and/or controlled release of the active agent upon exposure to an environment of use. Preferably, the pH modifying agent is in an effective amount to modifying the pH of the environmental fluid, e.g, gastric fluid, such that the dosage form delivers the drug having a pH dependent solubility over the desired period of time.

In certain embodiments, the dosage forms of the present invention comprise from about 1% by weight to about 90% by weight of an active agent and from about 0.1% by weight to about 25% by weight of an inorganic pH modifying agent. In other embodiments, the formulation comprises from about 20% by weight to about 75% by weight of an active agent and from about 0.5% by weight to about 15% by weight of an inorganic pH modifying agent. In other embodiments, the formulation comprises from about 30% by weight to about 50% by weight of an active agent and from about 1% by weight to about 8% by weight of an inorganic pH modifying agent. The inorganic pH modifying agent is preferably in an amount such that the release of the active agent from the formulation is not substantially affected by an increase in pH in the environment of use, resulting in an improved delivery of drugs which have a solubility which is subject to decline upon an increase in pH.

In certain embodiments when the active agent is clarithromycin, a pharmaceutically acceptable salt thereof, the dosage form provides a bioavailability which is from 80% to 125% of the bioavailability of a reference standard (Biaxin® Filmtab®; Biaxin® Granules; Biaxin® XL).

The pH modifying agent useful in the present invention preferably creates a pH below 7 in water at 5% w/v concentration. Preferably, the pH modifiers help to create an acidic micro environment to ensure release of drug from the dosage form at a higher pH environment in vitro and in vivo (i.e. the lower gastrointestinal tract).

The pH modifiers preferably provide a pH in a micro environment from greater than 3 to less than 7. More preferably from a pH of about 3.5 to about 5.5.

The pH modifying agent useful in certain embodiments of the present invention include, for example, sodium phosphate monobasic (NaH₂PO₄) and hydrated forms thereof, potassium phosphate monobasic and hydrated forms thereof, fumaric acid, glycyrrhizic acid, glycine and other acidic amino acids, cysteine hydrochloride and other basic amino acid salts, mixtures thereof, and the like.

In certain embodiments, the pH modifying agent is an organic pH modifier, e.g., citric acid, succinic acid, fumaric acid, malic acid, maleic acid, glutaric acid, lactic acid and mixtures thereof.

In certain embodiments, the pH modifier is an inorganic compound, for example, sodium phosphate monobasic (NaH₂PO₄) and hydrated forms thereof, potassium phosphate monobasic and hydrated forms thereof and mixtures thereof, and the like. The preferred pH modifier is sodium phosphate monobasic (NaH₂PO₄).

In certain other embodiment of the present invention, the drug and pH modifying agent, as well as any additional pharmaceutically acceptable ingredients can be incorporated into an immediate release or controlled release matrix.

In certain embodiments of the invention, oral administration of the controlled release dosage forms induce a lower mean fluctuation index in the plasma than the immediate release formulations of the drug while maintaining bioavailability substantially equivalent to that of the immediate release composition of the drug.

In certain embodiments of the invention, maximum peak concentrations of the drug (e.g., erythromycin derivative) after administration of the controlled release dosage forms are lower than those produced by the immediate release dosage forms, and area under the concentration-time curve and the minimum plasma concentration are substantially equivalent to that of the immediate release dosage forms.

In certain other embodiment, the present invention provides for a controlled release pharmaceutical dosage form comprising a macrolide antibiotic and a sufficient amount of a wax material to provide a controlled release of the macrolide antibiotic.

In certain embodiments of the present invention, the wax material is present in the dosage form in an amount from about 5% to about 95%, preferably from about 10% to about 35%.

The wax materials useful in the present invention include but are not limited to beeswax, white wax, emulsifying wax, hydrogenated vegetable oil, cetyl alcohol, stearyl alcohol, free wax acids such as stearic acid; esters of wax acids; propylene glycol monostearate and glyceryl monostearate; and carnauba wax. The wax material of the present invention preferably is a water insoluble wax material. Preferably, the wax material is a non-polymeric wax material. In certain preferred embodiments, the wax material is glyceryl monostearate, a glyceryl stearate, glyceryl palmitostearate, glyceryl behenate, stearyl alcohol, and stearic acid. Most preferably the wax material is glyceryl monostearate. Mixtures of any of the aforementioned wax materials may also be used.

In certain other embodiment, the present invention provides for a controlled release pharmaceutical dosage form comprising a macrolide antibiotic, a sufficient amount of a polymeric material to provide a controlled release of the macrolide antibiotic and an effective amount of at least one pH modifying agent to stabilize the macrolide antibiotic.

The pH modifying agent utilized for stabilizing the macrolide antibiotic can be an inorganic compound selected from the group discussed above or an organic compound such as cysteine hydrochloride and other basic amino acids, citric acid, fumaric acid, glutaric acid, glycyrrhizic acid, glycine and other acidic amino acids, lactic acid, malic acid, maleic acid, succinic acid and mixtures thereof and the like. The pH modifying agent is preferably an inorganic compound, most preferably sodium phosphate monobasic (NaH₂PO₄).

In embodiments which are stabilized, e.g., by inclusion of a sufficient amount of a pH modifying agent or other agent to stabilize the drug), the degradation of the drug (e.g., clarithromycin) is reduced by at least 5%, preferably at least 10%, more preferably at least 20% and most preferably at least 25%, at one or more times selected from 30 min., 60 min., 90 min., 120 min., 180 min., and 240 min. when placed in 0.01N HCL (pH 2.01).

In certain embodiments of the present invention, the polymeric material is a pharmaceutically acceptable acrylic polymer, including but not limited to acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamide copolymer, poly(methyl methacrylate), poly(methacrylic acid) (anhydride), methyl methacrylate, polymethacrylate, poly(methyl methacrylate), poly(methyl methacrylate) copolymer, polyacrylamide, aminoalkyl methacrylate copolymer, poly(methacrylic acid anhydride), and glycidyl methacrylate copolymers and any mixtures of the foregoing.

In certain embodiments of the present invention, the polymeric material is a pharmaceutically acceptable cellulosic material, e.g., alkylcellulose or a hydroxyalkylcellulose such as hydroxypropylmethylcellulose and mixtures of the foregoing.

In certain embodiments, the dosage forms of the present invention include at least one pharmaceutically acceptable excipient, such as an inert diluent. Diluents are widely used in the pharmaceutical arts. Examples of inert diluents are direct compression diluents including for example and without limitation, Di-Pac® (co-crystallized powder of highly modified dextrins (3% by weight) and sucrose) from Tate & Lyle, Baltimore, Md. Other direct compression diluents include Anhydrous lactose (Lactose N.F., anhydrous direct tableting) from Sheffield Chemical, Union, N.J. 07083; Elcems® M G-250 (Powdered cellulose, N.F.) from Degussa, D-600 Frankfurt (Main) Germany; Fast-Flo Lactose® (Lactose, N.F., spray dried) from Foremost Whey Products, Banaboo, Wis. 53913; Maltrin® (Agglomerated maltrodextrin) from Grain Processing Corp. Muscatine, Iowa 52761; Neosorb 60.® (Sorbitol, N.F., direct compression) from Roquette Corp., 645 5th Ave., New York, N.Y., 10022; Nu-Tab® (Compressible sugar, N.F.) from Ingredient Technology, Inc., Pennsauken, N.J. 08110; Polyplasdone XL® (Crospovidone, N.F., cross-linked polyvinylpyrrolidone) from GAF Corp., New York, N.Y. 10020; Primojel® (Sodium starch glycolate, N.F., carboxymethyl starch) from Generichem Corp., Little Falls, N.J. 07424; Spray-dried lactose® (Lactose N.F., spray dried) from Foremost Whey Products, Banaboo, Wis. 53913 and DMV Corp., Vehgel, Holland; and Sta-Rx 1500® (Starch 1500) (Pregelatinized starch, N.F., compressible) from Colorcon, Inc., West Point, Pa. 19486. In certain embodiments of the present invention, the diluent may or may not be mixed or partially mixed in an aqueous solution (e.g., water) prior to granulation.

In certain embodiments, the dosage forms can be prepared by wet-granulation. In certain embodiments the drug and stabilizer are wet granulated with the excipients. In other embodiments, at least a portion of the excipients are added to the mixture extragranularly. The term “extragranular” refers to material added to a pre-granulated material such that the mixture contains the added material intermixed with the pre-granulated material.

Alternatively, it is possible in certain embodiments to dry mix the ingredients without utilizing a wet granulation step. Thereafter, the mixture can be incorporated into a dosage form, e.g., a tablet or capsule.

In certain embodiments, a binder may be included in the dosage form. Examples of binders are acacia, cellulose derivatives (such as methylcellulose and carboxymethylcellulose, hydroxypropylmethylcellulose, hydroxypropylcellulose, hydroxyethylcellulose), gelatin, glucose, dextrose, xylitol, polymethacrylates, polyvinylpyrrolidone, starch paste, sucrose, sorbitol, pregelatinized starch, gum tragacanth, alginic acids and salts thereof such as sodium alginate, magnesium aluminum silicate, polyethylene glycol, guar gum, bentonites, and the like.

In the preparation of the dosage form, various solvents may be used to prepare the granules, preferably the solvents are aqueous solvents, e.g., water. In addition, various other diluents, excipients, lubricants, dyes, pigments, flavorants, colorants, dispersants, emulsifiers, glidants, plasticizers, etc. may be included in the formulations of the invention. The quantities of these additional materials will be sufficient to provide the desired effect to the desired formulation. Specific examples of pharmaceutically acceptable excipients that may be used to formulate oral dosage forms are described in the Handbook of Pharmaceutical Excipients, American Pharmaceutical Association (1986), incorporated by reference herein.

Examples of lubricants are magnesium stearate, glycerylbehaptate, polyethylene glycol, ethylene oxide polymers (for example, available under the registered trademark Carbowax from Union Carbide, Inc., Danbury, Conn.), sodium lauryl sulfate, magnesium lauryl sulfate, sodium oleate, sodium stearyl fumarate, DL-leucine, colloidal silica, and others as known in the art. The lubricant will be in the range of 0 to about 4 percent, and preferably 0 to about 2.5 percent by weight of the compressed, uncoated dosage form.

Examples of disintegrants are croscarmellose sodium, crospovidone, alginic acid, sodium alginate, methacrylic acid DVB, cross-linked PVP, microcrystalline cellulose, polacrilin potassium, sodium starch glycolate, starch, pregelatinized starch and the like. Preferred disintegrants are cross-linked polyvinylpyrrolidone (e.g. Kollidon CL), cross-linked sodium carboxymethylcellulose (e.g. Ac-Di-Sol), starch or starch derivatives such as sodium starch glycolate (e.g. Explotab®), or combinations with starch (e.g. Primojel), swellable ion-exchange resins, such as Amberlite IRP 88, formaldehyd-casein (e.g. Esma Spreng). Most preferably the disintegrant is sodium starch glycolate. The disintegrant may comprise approximately 0 to 20% of the total weight of the tablet.

Flavors incorporated in the composition may be chosen from synthetic flavor oils and flavoring aromatics and/or natural oils, extracts from plants leaves, flowers, fruits, and so forth and combinations thereof. These may include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil. Also useful as flavors are vanilla, citrus oil, including lemon, orange, grape, lime and grapefruit, and fruit essences, including apple, banana, pear, peach, strawberry, raspberry, cherry, plum, pineapple, apricot, and so forth. The amount of flavoring may depend on a number of factors including the organoleptic effect desired. Generally the flavoring will be present in an amount of from 0 to about 2 percent by weight based on the total tablet weight, when a flavor is used.

Colorants may include titanium dioxide and/or dyes suitable for food such as those known as F. D. & C, dyes and natural coloring agents such as grape skin extract, beet red powder, beta carotene, annato, carmine, turmeric, paprika, and so forth.

Additionally, an optional color coating may be used at a level in the range of 0-10% by weight which may be applied from a coating system such as Opadry® Yellow sold by Colorcon Corporation.

In certain embodiments of the present invention, the pharmaceutical composition may include other drugs in combination with one of the macrolide class of drugs mentioned above. For example, the macrolides erythromycin or clarithromycin may be formulated in combination with a preparation for standard therapy of gastritis, ulcers or gastroesophagal reflux disease (GERD), such as preparations containing anti-ulcer or anti-gastritis medicaments; e.g., selected among gastric secretion inhibiting compounds such as omeprazole, cimetidine, ranitidine, lansoprazole, pantoprazole, sucralfate, famotidine, or nizatidine, or antacids such as magnesium hydroxide, aluminum hydroxide, sodium carbonate, sodium hydrogen carbonate, simethicone or aluminum magnesium hydroxide or hydrate thereof (such as the monohydrate known as magaldrate). Additionally, the erythromycin or clarithromycin, pharmaceutical composition of the present invention may be adapted to be administered in combination with a preparation containing bismuth salts such as bismuth subcitrate, bismuth subsalicylate, bismuth subcarbonate, bismuth subnitrate or bismuth subgallate.

The following examples illustrate various aspects of the present invention. They are not to be construed to limit the claims in any manner whatsoever.

### EXAMPLE 1

Clarithromycin granules were prepared by wet granulation according to the following formulas listed in Table 1A. Forrmulation A is a control formulation without the addition of sodium phosphate monobasic in the granulating solution. Formulation B included sodium phosphate monobasic in the granulating solution in an amount equal to 7% of the resultant granules.

TABLE 1A

| | Ingredient | Formulation A | Formulation B |
|---|---|---|---|
| Granule | Clarithromycin | 56.5 | 56.5 |
| Formula | Compressible Sugar | 44.5 | 36.5 |
| (%) | NaH₂PO₄ | 0.0 | 7.0 |

Table 1B shows the effect of NaH₂PO₄ on pH Value of the Clarithromycin Granule Solution in 0.01N HCl (pH 2.01).

TABLE 1B

| | | 7% NaH₂PO₄ |
|---|---|---|
| Solution in 0.01N HC1 | 0% NaH₂PO₄ | Formulation |
| 5 mg/mL Clarithromycin | Formulation A | B(P00333) |
| pH | 2.46 | 2.59 |

Table 1C shows the effect of NaH₂PO₄ on pH Value of the Clarithromycin Granule Solution in Purified Water.

TABLE 1C

| Solution in Water | 0% NaH₂PO₄ | 7% NaH₂PO₄ |
|---|---|---|
| 5 mg/mL Clarithromycin | Formulation A | Formulation B |
| pH | 7.71 | 6.64 |

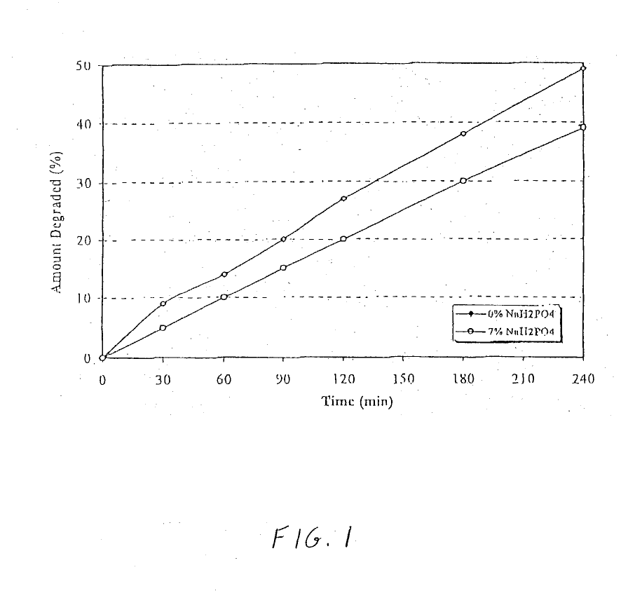

Table 1D shows the Effect of NaH₂PO₄ on Degradation of Clarithromycin Granules in 0.01N HCl (pH 2.01). These results are graphically presented depicted in FIG. 1.

TABLE 1D

| | 0% NaH₂PO₄ | 7% NaH₂PO₄ |
|---|---|---|
| Time | Formulation A | Formulation B |
| 0 | 0 | 0 |
| 30 | 9 | 5 |
| 60 | 14 | 10 |
| 90 | 20 | 15 |
| 120 | 27 | 20 |
| 180 | 38 | 30 |
| 240 | 49 | 39 |

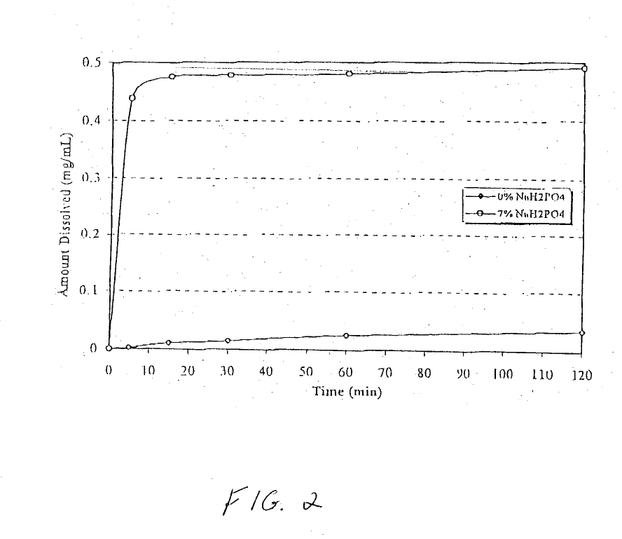

Table 1E shows the effect of NaH₂PO₄ on Dissolution of Clarithromycin Granules in Purified Water (pH 6.80). These results are graphically presented depicted in FIG. 2.

TABLE 1E

| | 0% NaH₂PO₄ | 7% NaH₂PO₄ |
|---|---|---|
| Time | Formulation A | Formulation B |
| 0 | 0 | 0 |
| 5 | 0.002 | 0.438 |
| 15 | 0.011 | 0.475 |
| 30 | 0.015 | 0.478 |
| 60 | 0.025 | 0.480 |
| 120 | 0.035 | 0.491 |
| | (SR1604-13C) | (P00333) |

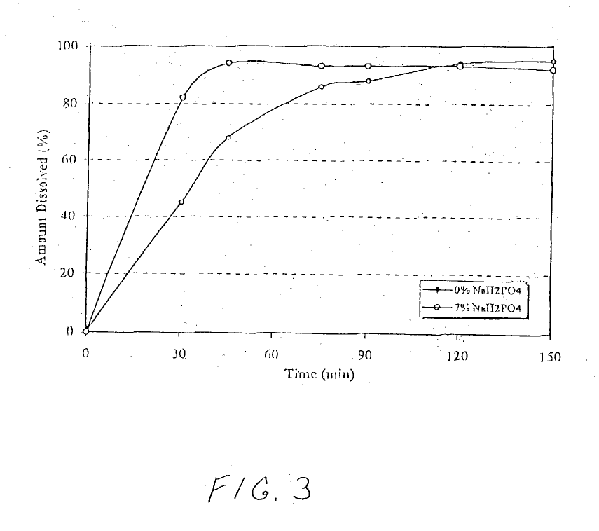

Table 1F shows the Effect of NaH₂PO₄ on Dissolution of Clarithromycin Granules in 0.01N HCl (ph 2.01). These results are graphically presented in FIG. 3.

TABLE 1F

| | 0% NaH₂PO₄ | 7% NaH₂PO₄ |
|---|---|---|
| Time | Formulation A | Formulation B |
| 0 | 0 | 0 |
| 30 | 45 | 82 |
| 45 | 68 | 94 |
| 75 | 86 | 93 |
| 90 | 88 | 93 |
| 120 | 94 | 93 |
| 150 | 95 | 92 |

### EXAMPLE 2

Clarithromycin extended release tablets were prepared according to the following formulas listed in Table 2A. Formulation C is a control formulation without the addition of sodium phosphate monobasic in the granulating solution. Formulation D included sodium phosphate monobasic in the granulating solution in an amount equal to 3% of the resultant formulation. Formulation E included sodium phosphate monobasic in the granulating solution in an amount equal to 6% of the resultant formulation.

TABLE 2A

| | | Formu- | Formu- | |
|---|---|---|---|---|
| | Ingredient | lation C | lation D | Formulation E |
| Tablet | Clarithromycin | 48.0 | 48.0 | 48.0 |
| Formula | Compressible Sugar | 37.0 | 34.0 | 31.0 |
| (%) | NaH₂PO₄ | 0.0 | 3.0 | 6.0 |
| | Glyceryl | 15.0 | 15.0 | 15.0 |
| | Monostearate | | | |

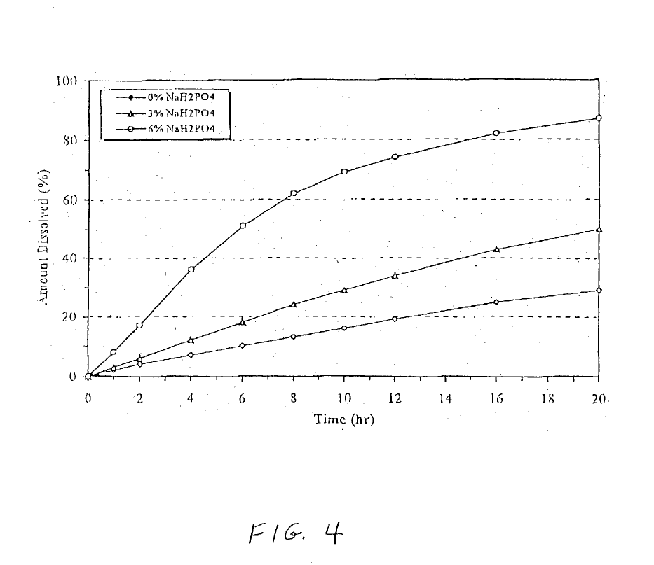

Table 2B shows an effect of NaH₂PO₄ on Dissolution of Clarithromycin ER Tablets in SIF(pH608)/0.5% Tween 80. These results are graphically presented in FIG. 4.

TABLE 2B

| | 0% NaH₂PO₄ | 3% NaH₂PO₄ | 6% NaH₂PO₄ |
|---|---|---|---|
| Time | Formulation C | Formulation D | Formulation E |
| 0 | 0 | 0 | 0 |
| 1 | 2 | 3 | 8 |
| 2 | 4 | 6 | 17 |
| 4 | 7 | 12 | 36 |
| 6 | 10 | 18 | 51 |
| 8 | 13 | 24 | 62 |
| 10 | 16 | 29 | 69 |
| 12 | 19 | 34 | 74 |
| 16 | 25 | 43 | 82 |
| 20 | 29 | 50 | 87 |

### EXAMPLE 3

Formulation for Clarithromycin XL Tablets, 500 mg IR Formulation

TABLE 3A
Clarithromycin Granules

| Ingredient: | mg/tablet | % | Wt. (kg) |
|---|---|---|---|
| Clarithromycin, USP | 500.00 | 56.5 | 11.600 |
| Compressible Sugar, NF (Di-Pac) | 353.98 | 40.0 | 8.212 |
| Sodium Phosphate Monobasic, USP | 30.97 | 3.5 | 0.719 |
| Purified Water, USP | | | 2.053 |
| | 884.96 | 100.0 | 20.531 |

Process: Sodium phosphage monobasic (11.6 kg) was blended with Di-pac (8.212 kg) in 5 cubic foot V-blender followed by blending with clarithromycin. The blend was wet granulated in a high shear granulator (FM-VG-100) by adding water (2.053 kg). The granules was then dried and screened to obtained clarithromycin granules at a LOD (loss on drying) no more than 3%.

TABLE 3B
Clarithromycin Tablets

| Ingredient: | mg/tablet | % | Wt (g) |
|---|---|---|---|
| Clarithromycin Granules | 884.96 | 85.0 | 12.75 |
| Microcrystalline Cellulose, NF (Avicel | 88.50 | 8.5 | 1.28 |
| PH102) | | | |
| Crospovidone, NF (Polyplasdone XL) | 52.06 | 5.0 | 0.75 |
| Explotab | — | — | — |
| Colloidal Silicon Dioxide, NF (Cab-O-Sil) | 5.21 | 0.5 | 0.08 |
| Magnesium Stearate, NF | 10.41 | 1.0 | 0.15 |
| Total | 1041.12 | 100.00 | 15.00 |

TABLE 3C
Clarithromycin Tablets

| Ingredient: | mg/tablet | % | Wt (g) |
|---|---|---|---|
| Clarithromycin Granules | 884.96 | 85.0 | 12.75 |
| Microcrystalline Cellulose, NF (Avicel | 88.50 | 8.5 | 1.28 |
| PH102) | | | |
| Crospovidone, NF (Polyplasdone XL) | 52.06 | 5.0 | 0.75 |
| Explotab | — | — | — |
| Colloidal Silicon Dioxide, NF (Cab-O-Sil) | 5.21 | 0.5 | 0.08 |
| Magnesium Stearate, NF | 10.41 | 1.0 | 0.15 |
| Total | 1041.12 | 100.00 | 15.00 |

Process: The clarithromycin granules were blended with the ingredients in Tables 3B and 3C. The blend was then compressed into tablets with tablet weight about 1141 mg.

### Example 4

Formulation for Clarithromycin XL Tablets, 500 mg IR Formulation

TABLE 4A
Clarithromycin Granules

| | mg/ | | |
|---|---|---|---|
| Ingredient: | tablet | % | Wt. (g) |
| Clarithromycin, USP | 500.00 | 76.0 | 25.00 |
| Compressible Sugar, NF(Di-Pac) | 98.68 | 15.0 | 4.93 |
| Compressible Sugar, NF(Di-Pac) (in solution) | 32.89 | 5.0 | 1.64 |
| Sodium Phosphate Monobasic, USP | 26.32 | 4.0 | 1.32 |
| Purified Water, USP | | | 4.93 |
| Total | 657.89 | 100.0 | 32.89 |

Process: Sodium phosphage monobasic (1.32 g) was first blended with Di-pac (4.93 g) followed by blending with clarithromycin. The blend was wet granulated by adding a solution of Di-Pac (1.64 g) in 4.93 g water. The granules was then dried and screened to obtain clarithromycin granules at a LOD no more than 3.

TABLE 4B
Clarithromycin Tablets

| Ingredient: | mg/tablet | % | Wt (g) |
|---|---|---|---|
| Clarithromycin Granules | 657.89 | 70.0 | 10.50 |
| Microcrystalline Cellulose, NF (Avicel | 220.86 | 23.5 | 3.53 |
| PH102) | | | |
| Croscarmelose (Ac-Di-Sol) | 46.99 | 5.0 | 0.75 |
| Explotab | — | — | — |
| Colloidal Silicon Dioxide, NF (Cab-O-Sil) | 4.70 | 0.5 | 0.08 |
| Magnesium Stearate, NF | 9.40 | 1.0 | 0.15 |
| Total: | 939.85 | 100.00 | 15.00 |

TABLE 4C
Clarithromycin Tablets

| Ingredient: | mg/tablet | % | Wt (g) |
|---|---|---|---|
| Clarithromycin Granules | 657.89 | 70.0 | 10.50 |
| Microcrystalline Cellulose, NF (Avicel | 220.86 | 23.5 | 3.53 |
| PH102) | | | |
| Croscarmelose (Ac-Di-Sol) | 46.99 | 5.0 | 0.75 |
| Explotab | — | — | — |
| Colloidal Silicon Dioxide, NF (Cab-O-Sil) | 4.70 | 0.5 | 0.08 |
| Magnesium Stearate, NF | 9.40 | 1.0 | 0.15 |
| Total: | 939.85 | 100.00 | 15.00 |

Process: Process: The clarithromycin granules were blended with the ingredients in Tables 4B and 4C. The blend was then compressed into tablets with tablet weight about 940 mg.

### EXAMPLE 5

Formulation for Clarithromycin XL Tablets, 500 mg IR Formulation

TABLE 5A
Clarithromycin Granules

| Ingredient: | mg/tablet | % | Wt. (g) |
|---|---|---|---|
| Clarithromycin, USP | 500.00 | 76.0 | 25.00 |
| Compressible Sugar, NF(Di-Pac) | 131.58 | 20.0 | 6.58 |
| Sodium Phosphate Monobasic, USP | 26.32 | 4.0 | 1.32 |
| Purified Water, USP | | | 5.92 |
| Total | 657.89 | 100.0 | 32.89 |

Process: Sodium phosphage monobasic (1.32 g) was first blended with Di-pac (4.93 g) followed by blending with clarithromycin. The blend was wet granulated by adding water. The granules was then dried and screened to obtain clarithromycin granules at a LOD no more than 3.

TABLE 5B
Clarithromycin Tablets

| Ingredient: | mg/tablet | % | Wt (g) |
|---|---|---|---|
| Clarithromycin Granules | 657.89 | 70.0 | 14.00 |
| Microcrystalline Cellulose, NF (Avicel | 220.86 | 23.5 | 4.70 |
| PH102) | | | |
| Croscarmelose (Ac-Di-Sol) | 46.99 | 5.0 | 1.00 |
| Colloidal Silicon Dioxide, NF (Cab-O-Sil) | 4.70 | 0.5 | 0.10 |
| Magnesium Stearate, NF | 9.40 | 1.0 | 0.20 |
| Total: | 939.85 | 100.00 | 20.00 |

TABLE 5C
Clarithromycin Tablets

| Ingredient: | mg/tablet | % | Wt (g) |
|---|---|---|---|
| Clarithromycin Granules | 657.89 | 70.0 | 14.00 |
| Microcrystalline Cellulose, NF (Avicel | 192.67 | 20.5 | 4.10 |
| PH102) | | | |
| Croscarmelose (Ac-Di-Sol) | 75.19 | 8.0 | 1.60 |
| Colloidal Silicon Dioxide, NF (Cab-O-Sil) | 4.70 | 0.5 | 0.10 |
| Magnesium Stearate, NF | 9.40 | 1.0 | 0.20 |
| Total: | 939.85 | 100.00 | 20.00 |

Process: Process: The clarithromycin granules were blended with the ingredients in Tables 5B and 5C. The blend was then compressed into tablets with tablet weight about 940 mg.

### EXAMPLE 6

A controlled release tablet containing 500 mg of clarithromycin and having the following formula was prepared.

Initially, clarithromycin granules were prepared via wet granulation and having the following formula listed in Table 6A.

TABLE 6A

| | mg/ | | Wt. |
|---|---|---|---|
| INGREDIENTS | tablet | % | (kg) |
| Clarithromycin Granules | | | |
| Clarithromycin, USP | 500.00 | 56.50 | 0.565 |
| Compressible Sugar, NF (Di-Pac) | 265.49 | 30.00 | 0.300 |
| Compressible Sugar, NF (Di-Pac) (in solution) | 88.50 | 10.00 | 0.100 |
| Sodium Phosphate Monobasic, USP | 30.97 | 3.50 | 0.035 |
| Purified Water, USP | | | 0.100 |
| Total: | 884.96 | 100.00 | 1.000 |

The granules where thereafter blended with the glyceryl monostearate to formulate a granular preparation which was then compressed into tablets using a tableting press and having the formula as shown in Table 6B below.

TABLE 6B

| Clarithromycin Extended-release Tablets: | mg/ | | |
|---|---|---|---|
| (Uncoated) | tablet | % | Wt. (kg) |
| Clarithromycin Granules | 884.96 | 85.0 | 0.600 |
| Glyceryl Monostearate, NF | 156.17 | 15.0 | 0.106 |
| (Eastman 600P) | | | |
| Total: | 1041.13 | 100.00 | 0.706 |

The tablets were thereafter coated with Opadry Yellow and Ethanol, SDA 3A 190 Proof, having the formula in Table 6C below.

TABLE 6C

| Color Coating | mg/tablet | % | Wt. (kg) |
|---|---|---|---|
| Clarithromycin Extended Release | 1041.13 | 97.00 | 2.910 |
| Tablets (Uncoated) | | | |
| Opadry Yellow | 32.20 | 3.00 | 0.090 |
| Ethanol, SDA 3A 190 Proof | * | | 0.810 |
| TOTAL | 1073.33 | 100.00 | 3.000 |

The final tablets formulated in Example 6 were compared, in vivo, to a currently marketed reference standard (Biaxin® XL) of the same dosage of clarithromycin, and gave the following fasting and fed results in Table 6D below:

TABLE 6D

| | Cₘₐₓ | AUC₀₋ₜ | Tₘₐₓ |
|---|---|---|---|
| Formulation | (μg/ml) | (μg · h/ml) | (h) |
| Fasting | | | |
| Example 6 | 1.43 ± 0.36 | 26.06 ± 10.83 | 5.25 ± 1.39 |
| Biaxin ® XL | 1.28 ± 0.43 | 26.40 ± 12.49 | 9.25 ± 5.12 |
| Fed: | | | |
| Example 6 | 2.79 ± 0.43 | 28.80 ± 6.34 | 4.50 ± 1.31 |
| Biaxin ® XL | 2.37 ± 0.30 | 31.44 ± 4.03 | 5.00 ± 1.51 |

### EXAMPLE 7

A controlled release tablet containing 500 mg of clarithromycin and having the following formula was prepared.

Initially, clarithromycin granules were prepared via wet granulation and having the following formula listed in Table 7A.

TABLE 7A

| | mg/ | | Wt. |
|---|---|---|---|
| INGREDIENTS | tablet | % | (kg) |
| Clarithromycin Granules | | | |
| Clarithromycin, USP | 500.00 | 56.5 | 0.565 |
| Compressible Sugar, NF (Di-Pac) | 234.51 | 26.5 | 0.265 |
| Compressible Sugar, NF (Di-Pac) (in solution) | 88.50 | 10.0 | 0.100 |
| Sodium Phosphate Monobasic, USP | 61.95 | 7.0 | 0.070 |
| Purified Water, USP | | | 0.100 |
| Total: | 884.96 | 100.00 | 1.000 |

The granules where thereafter blended with the glyceryl monostearate to formulate a granular preparation which was then compressed into tablets using a tableting press and having the formula as shown in Table 7B below.

TABLE 7B

| Clarithromycin Extended-release | | | |
|---|---|---|---|
| Tablets: (Uncoated) | mg/tablet | % | Wt. (kg) |
| Clarithromycin Granules | 884.96 | 85.0 | 0.600 |
| Glyceryl Monostearate, NF | 156.17 | 15.0 | 0.106 |
| (Eastman 600P) | | | |
| Total: | 1041.13 | 100.00 | 0.706 |

The tablets were thereafter coated with Opadry Yellow and Ethanol, SDA 3A 190 Proof, having the formula in Table 7C below.

TABLE 7C

| Color Coating | mg/tablet | % | Wt. (kg) |
|---|---|---|---|
| Clarithromycin Extended Release | 1041.13 | 97.00 | 2.910 |
| Tablets (Uncoated) | | | |
| Opadry Yellow | 32.20 | 3.00 | 0.090 |
| Ethanol, SDA 3A 190 Proof | * | | 0.810 |
| TOTAL | 1073.33 | 100.00 | 3.000 |

A controlled release tablet containing 500 mg of clarithromycin and having the following formula was prepared.

Initially, clarithromycin granules were prepared via wet granulation and having the following formula listed in Table 8A.

TABLE 8A

| | mg/ | | Wt. |
|---|---|---|---|
| INGREDIENTS | tablet | % | (kg) |
| Clarithromycin Granules | | | |
| Clarithromycin, USP | 500.00 | 56.50 | 0.565 |
| Compressible Sugar, NF (Di-Pac) | 265.49 | 30.00 | 0.300 |
| Compressible Sugar, NF (Di-Pac) (in solution) | 88.50 | 10.0 | 0.100 |
| Sodium Phosphate Monobasic, USP | 30.97 | 3.50 | 0.035 |
| Purified Water, USP | | | 0.100 |
| Total: | 884.96 | 80.00 | 1.000 |

The granules where thereafter blended with the glyceryl monostearate to formulate a granular preparation which was then compressed into tablets using a tableting press and having the formula as shown in Table 8B below.

TABLE 8B

| Clarithromycin Extended-release Tablets: | | | Wt. |
|---|---|---|---|
| (Uncoated) | mg/tablet | % | (kg) |
| Clarithromycin Granules | 884.96 | 80.0 | 0.600 |
| Glyceryl Monostearate, NF (Eastman 600P) | 221.24 | 20.0 | 0.150 |
| Total: | 1106.20 | 100.00 | 0.750 |

The tablets were thereafter coated with Opadry Yellow and Ethanol, SDA 3A 190 Proof, having the formula in Table 8C below.

TABLE 8C

| Color Coating | mg/tablet | % | Wt. (kg) |
|---|---|---|---|
| Clarithromycin Extended Release | 1106.20 | 97.00 | 2.910 |
| Tablets (Uncoated) | | | |
| Opadry Yellow | 34.21 | 3.00 | 0.090 |
| Ethanol, SDA 3A 190 Proof | * | | 0.810 |
| TOTAL | 1140.41 | 100.00 | 3.000 |

### EXAMPLE 9

A controlled release tablet containing 500 mg of clarithromycin and having the following formula was prepared.

Initially, clarithromycin granules were prepared via wet granulation and having the following formula listed in Table 9A.

TABLE 9A

| INGREDIENTS | mg/tablet | % | Wt. (kg) |
|---|---|---|---|
| Clarithromycin Granules | | | |
| Clarithromycin, USP | 500.00 | 56.50 | 0.565 |
| Compressible Sugar, NF (Di-Pac) | 280.97 | 31.75 | 0.3175 |
| Compressible Sugar, NF (Di-Pac) | 88.50 | 10.00 | 0.100 |
| (in solution) | | | |
| Sodium Phosphate Monobasic, USP | 15.49 | 1.75 | 0.0175 |
| Purified Water, USP | | | 0.100 |
| Total: | 884.96 | 100.00 | 1.000 |

The granules where thereafter blended with the glyceryl monostearate to formulate a granular preparation which was then compressed into tablets using a tableting press and having the formula as shown in Table 9B below.

TABLE 9B

| Clarithromycin Extended-release | | | |
|---|---|---|---|
| Tablets: (Uncoated) | mg/tablet | % | Wt. (kg) |
| Clarithromycin Granules | 884.96 | 85.0 | 0.600 |
| Glyceryl Monostearate, NF | 156.17 | 15.0 | 0.106 |
| (Eastman 600P) | | | |
| Total: | 1041.13 | 100.00 | 0.706 |

The tablets were thereafter coated with Opadry Yellow and Ethanol, SDA 3A 190 Proof, having the formula in Table 9C below.

TABLE 9C

| Color Coating | mg/tablet | % | Wt. (kg) |
|---|---|---|---|
| Clarithromycin Extended Release | 1041.13 | 97.00 | 2.910 |
| Tablets (Uncoated) | | | |
| Opadry Yellow | 32.20 | 3.00 | 0.090 |
| Ethanol, SDA 3A 190 Proof | * | | 0.810 |
| TOTAL | 1073.33 | 100.00 | 3.000 |

### EXAMPLE 10

A controlled release tablet containing 500 mg of clarithromycin and having the following formula was prepared.

Initially, clarithromycin granules were prepared via wet granulation and having the following formula listed in Table 10A.

TABLE 10A

| INGREDIENTS | mg/tablet | % | Wt. (kg) |
|---|---|---|---|
| Clarithromycin Granules | | | |
| Clarithromycin, USP | 500.00 | 56.5 | 0.565 |
| Compressible Sugar, NF (Di-Pac) | 265.49 | 30.0 | 0.300 |
| Compressible Sugar, NF (Di-Pac) | 88.50 | 10.0 | 0.100 |
| (in solution) | | | |
| Sodium Phosphate Monobasic, USP | 30.97 | 3.5 | 0.035 |
| Purified Water, USP | | | 0.100 |
| Total: | 884.96 | 100.00 | 1.000 |

The granules where thereafter blended with the glyceryl monostearate to formulate a granular preparation which was then compressed into tablets using a tableting press and having the formula as shown in Table 10B below.

TABLE 10B

| Clarithromycin Extended-release | | | |
|---|---|---|---|
| Tablets: (Uncoated) | mg/tablet | % | Wt. (kg) |
| Clarithromycin Granules | 884.96 | 80.0 | 0.450 |
| Glyceryl Monostearate, NF | 221.24 | 20.0 | 0.1125 |
| (Eastman 600P) | | | |
| Total: | 1106.20 | 100.00 | 0.5625 |

The tablets were thereafter coated with Opadry Yellow and Purified Water, USP, having the formula in Table 10C below.

TABLE 10C

| Color Coating | mg/tablet | % | Wt. (kg) |
|---|---|---|---|
| Clarithromycin Extended Release | 1106.20 | 97.00 | 2.910 |
| Tablets (Uncoated) | | | |
| Opadry Yellow | 34.21 | 3.00 | 0.090 |
| Purified Water, USP | * | | 0.810 |
| TOTAL | 1140.41 | 100.00 | 3.000 |

### EXAMPLE 11

A controlled release tablet containing 500 mg of clarithromycin and having the following formula was prepared.

Initially, clarithromycin granules were prepared via wet granulation and having the following formula listed in Table 11A.

TABLE 11A

| INGREDIENTS | mg/tablet | % | Wt. (kg) |
|---|---|---|---|
| Clarithromycin Granules | | | |
| Clarithromycin, USP | 500.00 | 56.50 | 0.565 |
| Compressible Sugar, NF (Di-Pac) | 265.49 | 30.00 | 0.300 |
| Compressible Sugar, NF (Di-Pac) | 88.50 | 10.00 | 0.100 |
| (in solution) | | | |
| Sodium Phosphate Monobasic, USP | 30.97 | 3.50 | 0.035 |
| Purified Water, USP | | | 0.100 |
| Total: | 884.96 | 100.00 | 1.000 |

The granules where thereafter blended with the glyceryl monostearate to formulate a granular preparation which was then compressed into tablets using a tableting press and having the formula as shown in Table 11B below.

TABLE 11B

| Clarithromycin Extended-release Tablets: | | | |
|---|---|---|---|
| (Uncoated) | mg/tablet | % | Wt. (kg) |
| Clarithromycin Granules | 884.96 | 75.0 | 0.400 |
| Glyceryl Monostearate, NF | 294.99 | 25.0 | 0.1333 |
| (Eastman 600P) | | | |
| Total: | 1179.95 | 100.0 | 0.5333 |

The tablets were thereafter coated with Opadry Yellow and Purified Water, USP, having the formula in Table 11C below.

TABLE 11C

| Color Coating | mg/tablet | % | Wt. (kg) |
|---|---|---|---|
| Clarithromycin Extended Release | 1179.95 | 97.00 | 2.910 |
| Tablets (Uncoated) | | | |
| Opadry Yellow | 36.49 | 3.00 | 0.090 |
| Purified Water, USP | * | | 0.810 |
| TOTAL | 1216.44 | 100.00 | 3.000 |

### EXAMPLE 12

A controlled release tablet containing 500 mg of clarithromycin and having the following formula was prepared.

Initially, clarithromycin granules were prepared via wet granulation and having the following formula listed in Table 12A.

TABLE 12A

| INGREDIENTS | mg/tablet | % | Wt. (g) |
|---|---|---|---|
| Clarithromycin Granules | | | |
| Clarithromycin, USP | 500.00 | 56.50 | 50.00 |
| Compressible Sugar, NF (Di-Pac) | 309.73 | 35.00 | 30.97 |
| Compressible Sugar, NF (Di-Pac) | 44.25 | 5.00 | 4.42 |
| (in solution) | | | |
| Sodium Phosphate Monobasic, USP | 30.97 | 3.50 | 3.10 |
| Purified Water, USP | | | 8.8 |
| Total: | 884.95 | 100.00 | 88.50 |

The granules where thereafter blended with the glyceryl monostearate to formulate a granular preparation which was then compressed into tablets using a tableting press and having the formula as shown in Table 12B below.

TABLE 12B

| Clarithromycin Extended-release | | | |
|---|---|---|---|
| Tablets: (Uncoated) | mg/tablet | % | Wt. (kg) |
| Clarithromycin Granules | 884.95 | 80.0 | 50.00 |
| Glyceryl Monostearate, NF | 221.24 | 20.0 | 12.50 |
| (Eastman 600P) | | | |
| Total: | 1106.19 | 100.00 | 62.50 |

A controlled release tablet containing 500 mg of clarithromycin and having the following formula was prepared.

Initially, clarithromycin granules were prepared via wet granulation and having the following formula listed in Table 13A.

TABLE 13A

| INGREDIENTS | mg/tablet | % | Wt. (g) |
|---|---|---|---|
| Clarithromycin Granules | | | |
| Clarithromycin, USP | 500.00 | 56.50 | 50.00 |
| Compressible Sugar, NF (Di-Pac) | 353.98 | 40.00 | 35.40 |
| Compressible Sugar, NF (Di-Pac) | 0.00 | 0.00 | 0.00 |
| (in solution) | | | |
| Sodium Phosphate Monobasic, USP | 30.97 | 3.50 | 3.10 |
| Purified Water, USP | | | 8.8 |
| Total: | 884.95 | 100.00 | 88.50 |

The granules where thereafter blended with the glyceryl monostearate to formulate a granular preparation which was then compressed into tablets using a tableting press and having the formula as shown in Table 13B below.

TABLE 13B

| Clarithromycin Extended-release Tablets: | | | |
|---|---|---|---|
| (Uncoated) | mg/tablet | % | Wt. (g) |
| Clarithromycin Granules | 884.95 | 80.0 | 50.00 |
| Glyceryl Monostearate, NF | 221.24 | 20.0 | 12.50 |
| (Eastman 600P) | | | |
| Total: | 1106.19 | 100.00 | 62.50 |

### EXAMPLE 14

A controlled release tablet containing 500 mg of clarithromycin and having the following formula was prepared.

Initially, clarithromycin granules were prepared via wet granulation and having the following formula listed in Table 14A.

TABLE 14A

| INGREDIENTS | mg/tablet | % | Wt. (kg) |
|---|---|---|---|
| Clarithromycin Granules | | | |
| Clarithromycin, USP | 500.00 | 56.5 | 11.600 |
| Compressible Sugar, NF (Di-Pac) | 353.98 | 40.0 | 8.212 |
| Compressible Sugar, NF (Di-Pac) | 0.00 | 0.00 | 0.000 |
| (in solution) | | | |
| Sodium Phosphate Monobasic, USP | 30.97 | 3.50 | 0.719 |
| Purified Water, USP | | | 2.053 |
| Total: | 884.96 | 100.00 | 20.531 |

The granules where thereafter blended with the glyceryl monostearate to formulate a granular preparation which was then compressed into tablets using a tableting press and having the formula as shown in Table 14B below.

TABLE 14B

| Clarithromycin Extended-release | | | |
|---|---|---|---|
| Tablets: (Uncoated) | mg/tablet | % | Wt. (kg) |
| Clarithromycin Granules | 884.96 | 80.0 | 8.800 |
| Glyceryl Monostearate, NF (Eastman | 221.24 | 20.0 | 2.200 |
| 600P) | | | |
| Total: | 1106.19 | 100.00 | 11.000 |

The tablets were thereafter coated with Opadry Yellow and Purified Water, USP, having the formula in Table 14C below.

TABLE 14C

| Color Coating | mg/tablet | % | Wt. (kg) |
|---|---|---|---|
| Clarithromycin Extended Release | 1106.19 | 97.0 | 2.910 |
| Tablets (Uncoated) | | | |
| Opadry Yellow | 34.21 | 3.0 | 0.090 |
| Purified Water, USP | * | | 0.660 |
| TOTAL | 1140.41 | 100.00 | 3.000 |

### EXAMPLE 15

A controlled release tablet containing 500 mg of clarithromycin and having the following formula was prepared.

Initially, clarithromycin granules were prepared via wet granulation and having the following formula listed in Table 15A.

TABLE 15A

| INGREDIENTS | mg/tablet | % | Wt. (kg) |
|---|---|---|---|
| Clarithromycin Granules | | | |
| Clarithromycin, USP | 500.00 | 56.50 | 11.600 |
| Compressible Sugar, NF (Di-Pac) | 353.98 | 40.00 | 8.212 |
| Compressible Sugar, NF (Di-Pac) | 0.00 | 0.00 | 0.000 |
| (in solution) | | | |
| Sodium Phosphate Monobasic, USP | 30.97 | 3.50 | 0.719 |
| Purified Water, USP | | | 2.053 |
| Total: | 884.96 | 100.00 | 20.531 |

The granules where thereafter blended with the glyceryl monostearate to formulate a granular preparation which was then compressed into tablets using a tableting press and having the formula as shown in Table 15B below.

TABLE 15B

| Clarithromycin Extended-release | | | |
|---|---|---|---|
| Tablets: (Uncoated) | mg/tablet | % | Wt. (kg) |
| Clarithromycin Granules | 884.96 | 75.0 | 8.250 |
| Glyceryl Monostearate, NF (Eastman | 294.99 | 25.0 | 2.750 |
| 600P) | | | |
| Total: | 1179.94 | 100.00 | 11.000 |

The tablets were thereafter coated with Opadry Yellow and Purified Water, USP, having the formula in Table 15C below.

TABLE 15C

| Color Coating | mg/tablet | % | Wt. (kg) |
|---|---|---|---|
| Clarithromycin Extended Release | 1179.94 | 97.0 | 9.700 |
| Tablets (Uncoated) | | | |
| Opadry Yellow | 36.49 | 3.0 | 0.300 |
| Purified Water, USP | * | | 2.200 |
| TOTAL | 1216.43 | 100.00 | 10.000 |

### EXAMPLE 16

A controlled release tablet containing 500 mg of clarithromycin and having the following formula was prepared.

Initially, clarithromycin granules were prepared via wet granulation and having the following formula listed in Table 16A.

TABLE 16A

| INGREDIENTS | mg/tablet | % | Wt. (kg) |
|---|---|---|---|
| Clarithromycin Granules | | | |
| Clarithromycin, USP | 500.00 | 56.50 | 0.565 |
| Compressible Sugar, NF (Di-Pac) | 296.46 | 33.50 | 0.335 |
| Compressible Sugar, NF (Di-Pac) | 88.50 | 10.00 | 0.100 |
| (in solution) | | | |
| Sodium Phosphate Monobasic, USP | 0.00 | 0.00 | 0.000 |
| Purified Water, USP | | | 0.100 |
| Total: | 884.96 | 100.00 | 1.000 |

The granules where thereafter blended with the glyceryl monostearate to formulate a granular preparation which was then compressed into tablets using a tableting press and having the formula as shown in Table 16B below.

TABLE 16B

| Clarithromycin Extended-release | | | |
|---|---|---|---|
| Tablets: (Uncoated) | mg/tablet | % | Wt. (kg) |
| Clarithromycin Granules | 884.96 | 85.0 | 0.600 |
| Glyceryl Monostearate, NF (Eastman | 156.17 | 15.0 | 0.106 |
| 600P) | | | |
| Total: | 1041.13 | 100.00 | 0.706 |

The tablets were thereafter coated with Opadry Yellow and Ethanol, SDA 3A 190 Proof, having the formula in Table 16C below.

TABLE 16C

| Color Coating | mg/tablet | % | Wt. (kg) |
|---|---|---|---|
| Clarithromycin Extended Release | 1041.13 | 97.0 | 2.910 |
| Tablets (Uncoated) | | | |
| Opadry Yellow | 32.20 | 3.0 | 0.090 |
| Ethanol, SDA 3A 190 Proof | * | | 0.810 |
| TOTAL | 1073.33 | 100.00 | 3.000 |

### EXAMPLE 17

Formulation for Clarithromycin XL Tablets, 500 mg Wax System with Glyceryl Monostearate and NaH₂PO₄

A controlled release tablet containing 500 mg of clarithromycin and having the following formula was prepared.

Initially, clarithromycin granules were prepared via wet granulation and having the following formula listed in Table 17A.

TABLE 17A
Granulation

| Ingredient: | mg/tablet | Total % | % | Wt. (g) |
|---|---|---|---|---|
| Clarithromycin, USP | 500.00 | 48.00 | 56.47 | 65.00 |
| Compressible Sugar, | 248.10 | 23.82 | 28.02 | 32.25 |
| NF (Di-Pac) | | | | |
| Compressible Sugar, | 137.33 | 13.18 | 15.51 | 17.85 |
| NF (Di-Pac) (for solution) | | | | |
| Sodium Phosphate Monobasic, | 0.00 | 0.00 | 0.00 | 0.00 |
| USP | | | | |
| Purified Water, USP | | | | 17.85 |
| Clarithromycin Granules | 885.43 | 85.00 | 100.00 | 115.11 |

The granules where thereafter blended with the glyceryl monostearate to formulate a granular preparation which was then compressed into tablets using a tableting press and having the formula as shown in Table 6B below.

TABLE 17B
Tableting

| | | | |
|---|---|---|---|
| Clarithromycin Granules | 885.43 | 85.0 | 15.00 |
| Glyceryl Monostearate, NF(Eastmen 600 P) | 156.25 | 15.0 | 2.65 |
| Clarithromycin Extended-release Tablets, | 1041.68 | 100.00 | 17.65 |
| 500 mg (Uncoated) | | | |

### EXAMPLE 18

The initial granules of Examples 6-17 can be incorporated into immediate release dosage forms as an alternative to combining the granules with controlled release excipients to form controlled release dosage forms. The immediate release dosage forms can further include pharmaceutically acceptable excipients known to one skilled in the art.

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

What is claimed is:

1. A controlled release oral dosage form comprising a matrix comprising:
a drug having a pH dependent solubility;
at least one wax material in an effective amount to provide a controlled release of said drug for at least 12 hours in an environment of use; and
at least one pH modifying agent.

2. The controlled release dosage form of claim 1 wherein said dosage form is suitable for once-a-day dosing.

3. The controlled release dosage form of claim 1 wherein said dosage form provides a therapeutic effect from about 12 to about 24 hours after administration to a human patient.

4. The controlled release dosage form of claim 1 wherein said dosage form provides a therapeutic effect for at least about 24 hours after administration to a human patient.

5. The controlled release oral dosage form of claim 1, wherein said drug is a macrolide antibiotic.

6. The controlled release oral dosage form of claim 5, wherein said macrolide antibiotic is clarithromycin or a pharmaceutically acceptable salt thereof.

7. The controlled release oral dosage form of claim 1, wherein said at least one wax material is selected from the group consisting of a glyceryl stearate, glyceryl palmitostearate, glyceryl behenate, glycerol monostearate, stearyl alcohol, and stearic acid.

8. The controlled release oral dosage form of claim 7, wherein said at least one wax material is glyceryl monostearate.

9. The controlled release oral dosage form of claim 1, wherein said pH modifying agent is an inorganic pH modifying agent.

10. The controlled release oral dosage form of claim 9, wherein said inorganic pH modifying agent is selected from the group consisting of sodium phosphate monobasic, hydrated forms thereof, potassium phosphate monobasic, hydrated forms thereof, and mixtures thereof.

11. The controlled release oral dosage form of claim 10, wherein said inorganic pH modifying agent is sodium phosphate monobasic.

12. The controlled release oral dosage form of claim 1, wherein said pH modifying agent is an organic pH modifying agent.

13. The controlled release oral dosage form of claim 12, wherein said organic pH modifying agent is selected from the group consisting of citric acid, succinic acid, fumaric acid, malic acid, maleic acid, glutaric acid, lactic acid, glycyrrhizic acid, glycine, cysteine hydrochloride and mixtures thereof.

14. The controlled release oral dosage form of claim 1, wherein pH modifying agent is selected from the group consisting of citric acid, succinic acid, fumaric acid, malic acid, maleic acid, glutaric acid, lactic acid, glycyrrhizic acid, glycine, cysteine hydrochloride, sodium phosphate monobasic, hydrated forms thereof, potassium phosphate monobasic, hydrated forms thereof, and mixtures thereof.

15. The controlled release oral dosage form of claim 1, wherein said dosage form is a tablet.

16. The controlled release oral dosage form of claim 1, wherein said matrix is divided into a plurality of units and contained within a capsule.

17. The controlled release oral dosage form of claim 1 wherein said drug, said wax material and said pH modifying agent are wet granulated without melting the wax to form a granulation and thereafter incorporating said granulation into a dosage form.

18. The controlled release oral dosage form of claim 17 wherein said granulation is compressed into a tablet.

19. The controlled release oral dosage form of claim 1 wherein said drug and said pH modifying agent are wet granulated to form a granulation and said granulation is mixed with said wax material without melting the wax to form a mixture which is incorporated into a dosage form.

20. The controlled release oral dosage form of claim 19 wherein said dosage form comprises a compressed tablet.

21. The controlled release oral dosage form of claim 1 which provides a bioavailability which is from 80% to 125% of the bioavailability of a reference standard (Biaxin® XL).

22. The controlled release oral dosage form of claim 1 which provides an mean AUC of from about 15 μg·h/ml to about 35 μg·h/ml based on administration of 500 mg clarithromycin.

23. The controlled release oral dosage form of claim 22 which provides a mean AUC of from about 20 μg·h/ml to about 30 μg·h/ml based on administration of 500 mg clarithromycin.

24. The controlled release oral dosage form of claim 23 which provides an mean AUC of from about 22 μg·h/ml to about 28 μg·h/ml based on administration of 500 mg clarithromycin.

25. The controlled release oral dosage form of claim 6 which provides a mean Cₘₐₓ from about 1 μg/ml to about 2 μg/ml based on administration of 500 mg clarithromycin under fasting conditions.

26. The controlled release oral dosage form of claim 6 which provides a mean Cₘₐₓ from about 2 μg/ml to about 3 μg/ml based on administration of 500 mg clarithromycin under fed conditions.

27. The controlled release oral dosage form of claim 6 which provides a mean AUC under fasted conditions which does not differ from the mean AUC under fed conditions by more than plus or minus 10%.

28. The controlled release oral dosage form of claim 1, wherein said dosage form provides a mean time to maximum plasma concentration (Tₘₐₓ) of the drug at from about 2 to about 8 hours after administration.

29. The controlled release oral dosage form of claim 1, wherein said dosage form provides a mean time to maximum plasma concentration (Tₘₐₓ) of the drug at from about 4 to about 6 hours after administration.

30. A method of preparing a controlled release oral dosage form comprising:
preparing a granulation comprising at least one drug having a pH dependent solubility and at least one pH modifying agent;
blending said granulation with at least one wax material in an effective amount to provide a controlled release of said drug for at least 12 hours in an environment of use, to form a mixture; and
incorporating said mixture into a dosage form.

31. The method of claim 30 wherein said mixture is compressed into a tablet with an optional pharmaceutical excipient.

32. The method of claim 30 wherein said granulation is a wet granulation.

33. The method of claim 30, wherein said drug is clarithromycin or a pharmaceutically acceptable salt thereof.

34. A method of preparing a controlled release oral dosage form comprising:
preparing a granulation comprising at least one drug having a pH dependent solubility, at least one pH modifying agent, and at least one wax material in an effective amount to provide a controlled release of said drug for at least 12 hours in an environment of use; and
incorporating said mixture into a dosage form.

35. The method of claim 34 wherein a portion of said wax material is introduced extragranularly.

36. The method of claim 34 wherein said granulation is compressed into a tablet with an optional pharmaceutical excipient.

37. A controlled release oral dosage form comprising:
clarithromycin or a pharmaceutically acceptable salt thereof and at least one controlled release excipient to provide a controlled release of said clarithromycin for at least 12 hours after administration to a human patient, wherein said dosage form provides a mean AUC under fasted conditions which does not differ from the mean AUC under fed conditions by more than plus or minus 10%.

38. A pharmaceutical dosage form comprising:
a drug having a pH dependent solubility; and
at least one inorganic compound in an effective amount to stabilize said drug.

39. The pharmaceutical dosage form of claim 38, wherein said dosage form is an oral dosage form.

40. The pharmaceutical dosage form of claim 39, wherein said dosage form provides an immediate release of said drug.

41. The pharmaceutical dosage form of claim 39, wherein said drug is a macrolide antibiotic.

42. The pharmaceutical dosage form of claim 39, wherein said macrolide antibiotic is clarithromycin, a pharmaceutically acceptable salt thereof or an active metabolite thereof.

43. The pharmaceutical dosage form of claim 42, wherein said active metabolite is 14-OH clarithromycin.

44. The pharmaceutical dosage form of claim 39, wherein said inorganic compound is a pH modifying agent.

45. The pharmaceutical dosage form of claim 44, wherein said inorganic compound is sodium phosphate monobasic.

46. The pharmaceutical dosage form of claim 39, wherein said dosage form is in the form of a tablet.

47. The pharmaceutical dosage form of claim 39, wherein said dosage form is in the form of a capsule.

48. The pharmaceutical dosage form of claim 39, wherein said drug and said inorganic compound are in the form of a granulation.

49. The pharmaceutical dosage form of claim 48, wherein said granulation is derived from a wet granulation.

50. The pharmaceutical dosage form of claim 48, wherein said granulation is derived from a dry granulation.

51. The pharmaceutical dosage form of claim 39, which provides a bioavailability which is from 80% to 125% of the bioavailability of a reference standard (Biaxin® XL).

52. The pharmaceutical dosage form of claim 39, which provides a bioavailability which is from 80% to 125% of the bioavailability of a reference standard (Biaxin® Filmtab®).

53. The pharmaceutical dosage form of claim 39, which provides a bioavailability which is from 80% to 125% of the bioavailability of a reference standard (Biaxin® Granules).

54. The pharmaceutical dosage form of claim 39, further comprising a sufficient amount of a polymeric material to provide a controlled release of said drug.

55. The pharmaceutical dosage form of claim 54, wherein said dosage form provides a controlled release of said drug for at least 12 hours in an environment of use.

56. The pharmaceutical dosage form of claim 54, wherein said dosage form provides a controlled release of said drug for at least 24 hours in an environment of use.

57. The pharmaceutical dosage form of claim 54, wherein said polymeric material comprises a cellulosic material.

58. The pharmaceutical dosage form of claim 54, wherein said polymeric material comprises an acrylic polymer.

59. The pharmaceutical dosage form of claim 39, wherein the inorganic compound provides a pH in a micro environment of from greater than 3 to less than about 7.

60. The pharmaceutical dosage form of claim 39, wherein the inorganic compound provides a pH in a micro environment of from greater than 3.5 less than about 5.5.

61. A controlled release pharmaceutical dosage form comprising:
a macrolide antibiotic and a sufficient amount of a wax material to provide a controlled release of said macrolide antibiotic.

62. The pharmaceutical dosage form of claim 61, wherein said macrolide antibiotic is erythromycin, a pharmaceutically acceptable salt thereof, an erythromycin derivative or mixtures thereof.

63. The pharmaceutical dosage form of claim 61, wherein said macrolide antibiotic is clarithromycin, a pharmaceutically acceptable salt thereof or an active metabolite thereof.

64. The pharmaceutical dosage form of claim 61, wherein said wax material is selected from the group consisting of beeswax, white wax, emulsifying wax, hydrogenated vegetable oil, cetyl alcohol, stearyl alcohol, free wax acids, esters of wax acids, propylene glycol monostearate, glyceryl monostearate, carnauba wax, glyceryl palmitostearate, glyceryl behenate, stearyl alcohol, stearic acid and combinations thereof.

65. A controlled release pharmaceutical dosage form comprising:
an antibiotic and a sufficient amount of glycerol monostearate to provide a controlled release of said antibiotic.

66. The pharmaceutical dosage form of claim 65, wherein said antibiotic a macrolide antibiotic.

67. The pharmaceutical dosage form of claim 66, wherein said macrolide antibiotic is clarithromycin, a pharmaceutically acceptable salt thereof or an active metabolite thereof.

68. The pharmaceutical dosage form of claim 65, wherein said macrolide antibiotic is erythromycin, a pharmaceutically acceptable salt thereof, an erythromycin derivative or mixtures thereof.

69. A controlled release pharmaceutical dosage form comprising:
a macrolide antibiotic;
a sufficient amount of a polymeric material to provide a controlled release of said macrolide antibiotic; and
an effective amount of at least one inorganic compound to stabilize said macrolide antibiotic.

70. A controlled release pharmaceutical dosage form comprising:
a macrolide antibiotic;
a sufficient amount of a polymeric material to provide a controlled release of said macrolide antibiotic; and
an effective amount of at least one organic compound to stabilize said macrolide antibiotic, said organic compound selected from the group consisting of citric acid, succinic acid, fumaric acid, malic acid, maleic acid, glutaric acid, lactic acid.

71. The pharmaceutical dosage form of claim 69, wherein said compound is sodium phosphate monobasic.

72. The pharmaceutical dosage form of claim 69, wherein the inorganic compound provides a pH in a micro environment of from greater than 3 to less than about 7.

73. The pharmaceutical dosage form of claim 69, wherein the inorganic compound provides a pH in a micro environment of from greater than 3.5 less than about 5.5.

74. A method of preparing a pharmaceutical dosage form comprising:
preparing a granulation comprising at least one drug having a pH dependent solubility and an effective amount of at least one inorganic compound; and
incorporating said granulation into a dosage form.

75. The method of claim 74, wherein said granulation is compressed into a tablet with an optional pharmaceutical excipient.

76. The method of claim 74, wherein said granulation is a wet granulation.

77. The method of claim 74, wherein said granulation is a dry granulation.

78. The method of claim 74, wherein said drug is clarithromycin, a pharmaceutically acceptable salt thereof or active metabolite thereof.

79. A method of preparing a pharmaceutical dosage form comprising:
preparing a granulation comprising a macrolide antibiotic and a sufficient amount of a wax material to provide a controlled release of said macrolide antibiotic; and
incorporating said granulation into a dosage form.

80. A method of preparing a pharmaceutical dosage form comprising:
preparing a granulation comprising an antibiotic and a sufficient amount of glycerol monostearate to provide a controlled release of said antibiotic; and incorporating said granulation into a dosage form.

81. A method of preparing a pharmaceutical dosage form comprising preparing a granulation comprising a macrolide antibiotic; a sufficient amount of a polymeric material to provide a controlled release of said drug and an effective amount of at least one compound to stabilize said drug; and incorporating said granulation into a dosage form.

82. A pharmaceutical dosage form comprising clarithromycin or a pharmaceutically acceptable salt thereof and an effective amount of a stabilizer such that the degradation of the clarithromycin is reduced by at least 5% at one or more times selected from 30 min., 60 min., 90 min., 120 min., 180 min., and 240 min. when placed in 0.01N HCL (pH 2.01).
